(19) United States
(12) Patent Application Publication
(10) Pub. No.: US 2010/0132431 A1
(43) Pub. Date: Jun. 3, 2010

(54) METHOD FOR TESTING AND CALIBRATING A CAPACITIVE FLEX FUEL SENSOR

(76) Inventors: Norberto Hernandez, Chihuahua (MX); Carlos A. Urquidi, Chihuahua (MX); Esau Aguinaga, Chihuahua (MX)

Correspondence Address: DELPHI TECHNOLOGIES, INC;LEGAL STAFF - M/C 483-400-402, 5725 DELPHI DRIVE, PO BOX 5052, TROY, MI, 48007, US

(21) Appl. No.: 12/326,416

(22) Filed: Dec. 2, 2008

(51) Int. Cl. G01N 33/22

(52) U.S. Cl. 73/1.06

(57) ABSTRACT

A method for calibrating a flex fuel sensing apparatus having a fuel passage involves the use of a generally solid test blank having a size and shape configured to match that of the fuel passage. The test blank comprises material having a known, predetermined dielectric constant. The test blank is inserted into the fuel passage where the dielectric constant of the test blank is operative to simulate the presence of various gasoline and ethanol fuel blends. The test blank may also comprise a deformable/compressible solid such as rubber where the test blank is compressed during calibration to eliminate air gaps or the like, improving accuracy. The use of the solid test blank reduces complexity compared to using actual gasoline/ethanol blends for calibration as well as reduces post-calibration cleanup.

### CROSS-REFERENCE TO RELATED APPLICATIONS

This application is related to co-pending U.S. application Ser. No. ______ filed ______, 2008 entitled “FUEL SENSOR (Docket No. DP-317807), now pending, owned by the common assignee of the present invention, the disclosure of which is hereby incorporated by reference herein in its entirety.

### TECHNICAL FIELD

The present invention relates generally to sensors, more particularly to a fuel sensor having sensing plates that do not obstruct a fuel passage, and still more particularly to a method for testing and calibrating such sensors.

### BACKGROUND OF THE INVENTION

Due to the fact that ethanol is a renewable fuel, and for other reasons as well, the use of ethanol and ethanol blends (i.e., ethanol and gasoline) continues to grow. For example, flexible fuel vehicles are known that are designed to run on gasoline as a fuel or a blend of up to 85% ethanol (E85). Properties of such fuels, such as its conductivity or dielectric constant, can be used to determine the concentration of ethanol (or other alternate fuel) in the gasoline/alternate fuel blend and can also be used to determine the amount of water mixed in with the fuel. Experimental data shows that the fuel dielectric constant is directly proportional to the ethanol concentration but relatively insensitive to water contamination, provided that the water concentration is below about 1% since the dielectric constant of water is around 80 at 25° C. (i.e., surveys show that the water concentration on most U.S. Flex fuel stations is below 1%). On the other hand, fuel conductivity is very sensitive to water concentration. For example, ethanol has a dielectric constant of around 24 at 25 degrees Celsius while gasoline has a dielectric constant of around 2 at the same temperature. Determining the properties of such fuels is important for operation of a motor vehicle since an engine controller or the like can use the information regarding the composition, quality, temperature and other properties of the fuel to adjust air/fuel ratio, ignition timing and injection timing, among other things. Additionally, increasingly strict emissions-compliance requirements have only further strengthened the need for an accurate flexible fuel sensor.

Fuel passage obstruction is a shortcoming of conventional fuel sensors, particularly capacitance-based approaches. More specifically, to measure the capacitance of the fuel, conventional sensors are known to use plates with different shapes, but in all such applications these plates are inside the fuel line (i.e., the fuel passage). This makes the construction of such sensors more complex and poses a potential for obstructing the fuel flow. Additionally, this approach imposes stricter requirements to protect the plates from corrosion by the ethanol.

Co-pending application (Attorney Docket No. DP-317807) disclose an obstruction-less fuel property sensor that measures, for example, the ethanol concentration in the fuel, which is then used by the engine control unit for controlling the engine to accommodate the fuel with the varying ethanol levels. As known, different blends have varying energy content levels. The sensor in the co-pending application measures capacitance across the fuel that flows through the sensor and from which a corresponding dielectric constant can be derived. The sensor then correlates the dielectric constant with an ethanol concentration. The output of the sensor may be, in one embodiment, proportional to the dielectric constant of the media under measurement, and which may vary between about 2 and 24 (as described above).

In the manufacturing process of this kind of flex fuel sensor, important steps involve calibration and final testing. To perform the calibration, one approach involves exposing the sensor to a series of different fuel formulations containing varying (but known) ethanol concentrations, like what which would be encountered during the service life of the sensor. For each fuel formulation, a respective reading would be taken from the sensor. The readings would be used to arrive at a calibration factor (or a curve or a map in some instances) that could be used to interpret or convert the actual sensor readings into a more accurate indication of ethanol concentration. However, a drawback of such an approach is that using fuels in the manufacturing process (even for testing) complicates the handling and the cleaning of the sensor, as well as requires increased levels of care (e.g., safety measures) in the handling of the fuels. Overall, this introduces extra expensive.

Another approach involves the use of other liquids to simulate different gasoline and ethanol blends of fuels (e.g., like various oils). However, a drawback of this approach is that it will still require a special step to clean the sensor and/or related parts after the calibration and test phases. The step of cleaning itself may involve or require the use of solvents, which can complicate the overall process as much as the use of fuel blends. In sum, the use of oils or the like do not provide any appreciable improvement over use of fuels.

There is therefore a need for a method for calibration and testing of a fuel property sensor that minimizes and/or eliminates one or more of the problems described above.

### SUMMARY OF THE INVENTION

The present invention solves one or more of the problems described above in connection with calibrating/testing a fuel sensor by employing a test blank formed of a solid material having a known dielectric constant. The test blank is formed with a size and shape configured to match that of the fuel passageway in the fuel sensor and is used in place of actual fuel during calibration and final testing. The invention reduces the cost of the equipment needed for calibration/testing, its operating cost, as well as eliminating previously required related costs pertaining to safety measures and sensor cleaning.

A method of calibrating a fuel property sensor having a fuel passage includes a number of steps. The first step involves inserting a test blank into the fuel passage wherein the blank comprises material having a predetermined dielectric constant. Preferably, the test blank is configured in size and shape to correspond to the fuel passage, thereby substantially occupying the passage. The next step involves calibrating the fuel property sensor while the blank remains in the fuel passage.

In an alternate embodiment, the method further includes the step of compressing the test blank while it is in the fuel passage so as to substantially eliminate “air gaps” due to irregularities in either the inner surface of the fuel passage or the outer surface or geometry and/or size of the test blank. Minimizing or eliminating these “air gaps” allows for a more accurate calibration.

### BRIEF DESCRIPTION OF THE DRAWINGS

The present invention will now be described by way of example, with reference to the accompanying drawings:

FIG. 1 is a top, perspective view of an embodiment of an obstruction-less in-line flexible fuel sensing apparatus shown in a preferred environment.

FIG. 2 is a top, perspective view of the fuel sensing apparatus of FIG. 1 arranged for a calibration and testing phase of a manufacturing process.

FIG. 3 is a cross-sectional view of the fuel sensing apparatus of FIG. 2 taken substantially along lines 3-3 and having a generally non-deformable test blank inserted in the fuel passage.

FIG. 4 is a cross-sectional view of the fuel sensing apparatus of FIG. 2 taken substantially along lines 3-3 having a generally deformable test blank inserted in the fuel passage.

FIG. 5 is a flowchart diagram showing a method of calibrating a fuel property sensing apparatus in accordance with the present invention.

### DETAILED DESCRIPTION OF THE PREFERRED EMBODIMENT

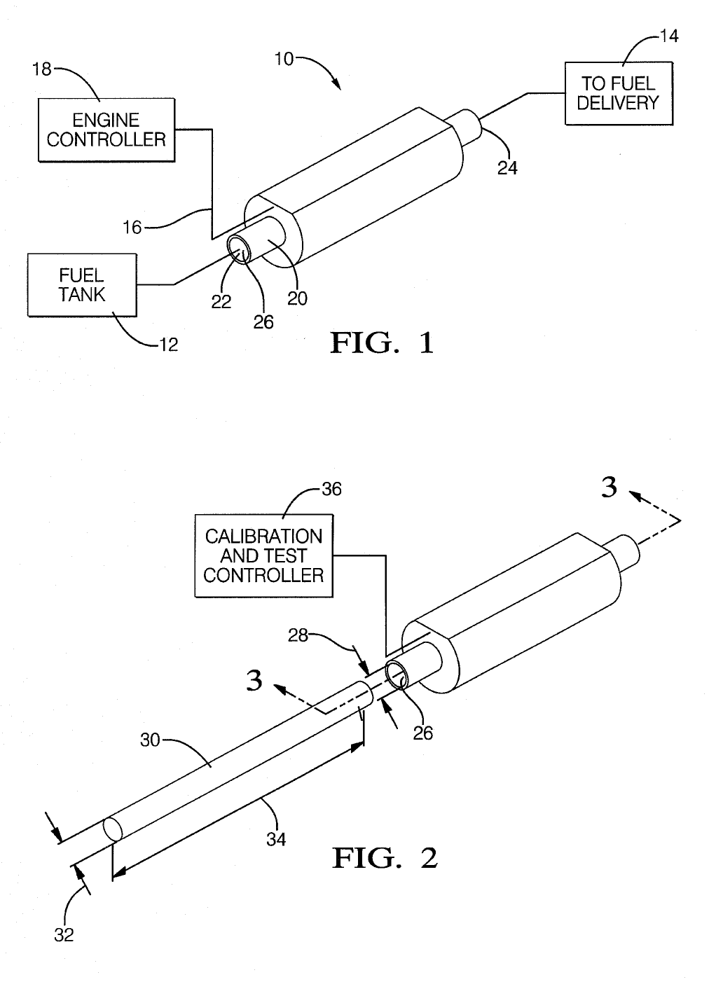

Referring now to the drawings wherein like reference numerals are used to identify identical components in the various views, FIG. 1 is a perspective view of a sensing apparatus 10 for sensing one or more properties of a fuel, such as a dielectric constant of a gasoline/ethanol blend. The sensing apparatus 10 (sometimes referred to herein as a “sensor”), as shown, is an in-line type fuel sensing apparatus that is coupled between a source of fuel, such as a fuel tank 12, and a destination, such as various fuel delivery apparatus 14 associated with an automotive vehicle internal combustion engine (not shown). The sensing apparatus 10, generally, includes a pair of sensing plates (not shown) surrounding an inner tube, in a concentric manner, which are connected to a closely-located electrical circuit with signal processing capability so as to generate an output signal 16. The sensing plates around the inner tube will form a capacitor. The material between the plates includes a fixed portion, namely the tube walls, which have a fixed dielectric constant. However, the dielectric constant of the fuel flowing through the fuel line will vary, depending on the composition of the fuel itself. The total effective capacitance will be mainly driven by the variable portion. The circuit will measure the capacitance for purposes of generating the signal 16.

The output signal 16 is indicative of one or more sensed physical properties of the fuel, such as dielectric constant or conductivity. The output signal 16 may then be provided to, for example only, an electronic engine controller 18 or the like for use in engine control, as described above. The sensing apparatus 10 further includes a tube 20 formed of fuel resistant material (e.g., acetal) having a fuel inlet 22, a fuel outlet 24 and a fuel passage 26 formed therebetween. Further details of the kind of fuel sensing apparatus to which the inventive method for calibrating and final testing may find useful application may be seen by reference to co-pending U.S. patent application entitled “FUEL SENSOR” (attorney docket no. DP-317807) referenced above.

FIG. 2 is a top, perspective view of the fuel sensing apparatus 10 of FIG. 1, arranged for a calibration and testing phase of a manufacturing process. The fuel passage 26, in the illustrated embodiment, is generally cylindrical in shape and has a first diameter 28 associated therewith. FIG. 2 also shows a test blank 30, which is configured to simulate the dielectric properties of a gasoline/ethanol fuel blend and to be used in place of the fuel blend for calibration/testing purposes. As shown, the test blank 30 is configured in size and shape to correspond to and match the fuel passage 26 (i.e., substantially fill the fuel passage 26). As shown, where the fuel sensor 10 is cylindrical in shape, the test blank 30 will also be cylindrical in shape, having a second diameter 32 and a length 34, which match the respective diameter (i.e., first diameter 26) and length of the fuel passage 26. It should be understood that other shapes and geometries are within the spirit and scope of the invention. In one embodiment, the test blank 30 is formed of solid materials with different dielectric constants, including but not limited to, for example only, paper, Portland cement, various resin-based solids (e.g., DELRIN® brand acetal resin, commercially available from E.I. DuPont DeNemours & Co. of Wilmington, Del., USA) for low dielectric constants or ceramic for high dielectric constants.

In addition, FIG. 2 shows a calibration and test controller 36 configured to perform the calibration and final testing in accordance with known strategies. The controller 36 may comprise conventional apparatus known in the art for such purposes. The controller 36 is configured, among other things, to cause the sensing apparatus 10 to apply an excitation signal across the sensing plates (not shown) contained in the sensing apparatus 10 and to cause the sensing apparatus 10 to measure the resulting induced signal and provide an output indicative of the dielectric constant of the media-under-test (i.e., here, the test blank 30). A resulting calibration factor (or curve/map as the case may be for multiple, different points/test blanks 30) may be stored in the sensing apparatus 10 at the end of the calibration phase for use by the sensing apparatus 10 during its service life. Alternatively, the controller 36 may store the resulting calibration factor (or curve/map, as the case may be) in a separate database or the like for ultimate end-use by the engine controller 18 (FIG. 1) when this particular, calibrated sensing apparatus 10 is deployed in the field (i.e., the engine controller 18 may be configured to receive an initial or updated fuel sensor calibration upon installation of this particular calibrated sensing apparatus 10). There are numerous other approaches known in the art for using a calibration factor or map once determined.

In sum, the test blank 30 fits in the fuel passage 26 of the sensing apparatus 10 to simulate the different fuel blends required in the calibration and test. This inventive approach using a solid test blank greatly simplifies the process while at the same time provides an approach that eliminates the need for extensive post-calibration cleaning.

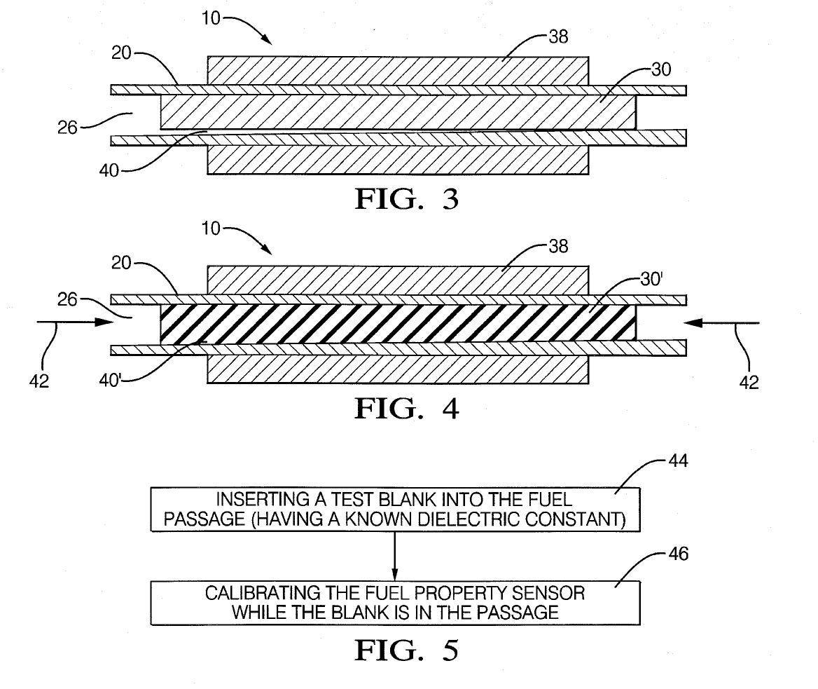

FIG. 3 is a cross-sectional view of the fuel sensing apparatus 10 of FIG. 2 taken substantially along lines 3-3 and having a generally non-deformable test blank 30 inserted in the fuel passage 26. FIG. 3 further shows the location of a capacitive sensing structure 38, which may comprise a pair of sensing electrodes (not shown) outlying the fuel passage tube 20. As shown, due to imperfections in either the tube 20 or in the test blank 30, unfilled gaps 40 may exist in the fuel passage 26, lessening the accuracy of the calibration. This is because the unfilled gap 40 will introduce a different dielectric constant than that of the solid test blank 30. Of course, when fuel flows through the fuel passage 26, its fluidity will cause it to occupy the entire volume in the fuel passage 26 (i.e., no air gaps). Accordingly, simulating this complete fill aspect is also desirable.

FIG. 4 is a cross-sectional view of the fuel sensing apparatus 10 of FIG. 2 taken substantially along lines 3-3, and having a generally solid but deformable test blank 30′ (e.g., rubber) inserted in the fuel passage 26 (the controller 36 has been omitted for clarity). As shown, the calibration and test setup further includes means 42 for compressing the inserted test blank 30′. The compressing means 42 is preferably configured to apply a compressing force to opposing ends of the test blank 30′. In one embodiment, the compressing means 42 may include an automated machine with pneumatic pistons that are coupled to an air pressure regulator or other source of compressed air. Other variations are possible. Through this extra step, the previously-evident unfilled gaps 40 are now filled, as indicated by reference numeral 40′. The elimination of the gaps 40 improves the accuracy of the calibration by eliminating the air gaps that have a dielectric constant different from that of the test blank 30′.

FIG. 5 is a flowchart diagram showing a method of calibrating a fuel property sensing apparatus 10 in accordance with the present invention. The method begins in step 44.

In step 44, the test blank 30 (or 30′) is inserted in the fuel passage 26 of the fuel sensing apparatus 10. The test blank 30 (or 30′) is preferably solid and comprises material having a predetermined, known dielectric constant. The method proceeds to step 46.

In step 46, the controller 36 is configured to calibrate the fuel sensing apparatus 10 while the test blank 30 (or 30′) is in the fuel passage 26. In one embodiment, the calibrating step includes the step of applying an excitation signal of a predetermined, known character while measuring the resulting, induced signal. From the excitation and induced signals, a capacitance can be derived, from which a test-based dielectric constant of the media-under-test (i.e., test blank 30 or 30′) can be computed, all as previously described in the U.S. application entitled “FUEL SENSOR” (docket no. DP-317807), referred to above and incorporated herein by reference. The controller 38 may be further configured to calculate a calibration factor based on the test-based dielectric constant of the media-under-test, on the one hand, and the predetermined, known dielectric constant of the media-under-test, on the other hand. Finally, the method may involve storing the calculated calibration factor (or curve/map as the case may with multiple test blanks of different dielectric constants), in accordance with at least one calibration usage strategy known in the art. Two exemplary approaches were described above, however, it should be understood that those are exemplary only and not limiting in nature.

While the invention has been described in connection with what is presently considered to be the most practical and preferred embodiment, it is to be understood that the invention is not to be limited to the disclosed embodiments but, on the contrary, is intended to cover various modifications and equivalent arrangements included within the spirit and scope of the appended claims, which scope is to be accorded the broadest interpretation so as to encompass all such modifications and equivalent structures as is permitted under the law

1. A method of calibrating a fuel property sensing apparatus having a fuel passage comprising the steps of:
(A) inserting a test blank into the fuel passage wherein the blank comprises material having a predetermined dielectric constant; and
(B) calibrating the fuel property sensing apparatus sensor while said blank is in said passage.

2. The method of claim 1 further including the step of:
providing the test blank with a size and shape corresponding to the fuel passage.

3. The method of claim 2 wherein the fuel passage and the test blank are cylindrical.

4. The method of claim 1 wherein said material comprises a solid material selected from the group comprising paper, Portland cement, Acetal homopolymer, and ceramic.

5. The method of claim 2 wherein said test blank comprises a material having elastic properties, and wherein said method further includes the step of:
compressing the test blank so as to substantially occupy said fuel passage.

6. The method of claim 5 wherein said material comprises rubber.

7. The method of claim 1 wherein said predetermined dielectric constant is between about 2 and 24.

8. The method of claim 1 wherein said step of calibrating includes the sub-steps of:
applying an excitation signal and measuring a resulting induced signal;
determining a test dielectric constant based on the excitation signal and the induced signal;
calculating a calibration factor based on the predetermined dielectric constant and the test dielectric constant.

9. The method of claim 8 wherein said calibrating step further including the substep of:
storing the calibration factor.
